(19) United States
(12) Patent Application Publication
(10) Pub. No.: US 2026/0196358 A1
(43) Pub. Date: Jul. 9, 2026

(54) System and Method for Population Health Monitoring Using Mobile-Based Walking and Balance Data Collection

(71) Applicant: Stanley G. Van Meter, II, Lake Mary, FL (US)

(72) Inventor: Stanley G. Van Meter, II, Lake Mary, FL (US)

(21) Appl. No.: 19/426,865

(22) Filed: Dec. 19, 2025

Related U.S. Application Data
(60) Provisional application No. 63/736,699, filed on Dec. 20, 2024.

(51) Int. Cl. G16H 50/50

(52) U.S. Cl. CPC G16H 50/50

(57) ABSTRACT

A system and method for collecting and analyzing real-time walking and balance data from mobile device users through an opt-in mechanism integrated with smartphone operating systems (iOS and Android). Users voluntarily share their mobility data, including gait speed, stride length, and balance stability, which is anonymized and analyzed using machine learning algorithms. The system provides personalized health insights, such as real-time warnings about fall risks, and generates population-level health trend reports. Aggregated, anonymized data helps public health organizations and healthcare providers identify emerging trends, such as regional increases in fall risks or correlations between medication use and mobility issues. This dual-purpose system bridges individual health monitoring and population-level public health analysis, offering proactive interventions to improve well-being. The system ensures privacy compliance while enabling early detection and prevention of health risks for both individuals and broader communities.

### FEDERALLY SPONSORED RESEARCH

Not Applicable

### SEQUENCE LISTING OR PROGRAM

Not Applicable

### TECHNICAL FIELD OF THE INVENTION

The present invention relates to mobile health data collection and analysis, with a focus on utilizing opt-in walking and balance data from users through smartphone operating systems (iOS and Android). Mor specifically, the present invention enables the tracking of population health trends and provides individualized health warnings, improving public and personal health outcomes through real-time data aggregation and analysis.

### BACKGROUND OF THE INVENTION

The present invention relates to the field of mobile health data collection and analysis, with a focus on leveraging walking and balance data from users'smartphones to provide both individualized health insights and population-level health trend analysis. Using smartphone operating systems (iOS and Android), this invention enables users to opt-in and share their walking and balance data, which is collected and analyzed to identify mobility risks, such as falls, and broader public health patterns.

Current health monitoring systems typically focus on individual users without the ability to aggregate real-time data across large populations. This limitation hinders the identification of emerging public health trends and the implementation of timely interventions. There is a growing need for systems that not only monitor individual health metrics but also analyze population-level data to identify risks, improve public health outcomes, and provide proactive, personalized warnings to users. The invention addresses this gap by combining real-time data collection, advanced analytics, and secure anonymization to deliver insights for both individuals and public health stakeholders.

### SUMMARY OF THE INVENTION

The present invention provides a system and method for collecting and analyzing real-time walking and balance data from mobile device users through an opt-in mechanism integrated with smartphone operating systems (iOS and Android). This system addresses the dual need for individualized health monitoring and population-level health trend analysis by enabling users to voluntarily share their mobility data, such as gait speed, stride length, and balance stability. The collected data is analyzed using machine learning algorithms to provide personalized health insights, including real-time warnings about fall risks or mobility issues.

At the population level, the system aggregates anonymized data to identify trends across specific demographics or regions, such as seasonal increases in fall risk or mobility declines in certain areas. These insights are shared with public health organizations, healthcare providers, and insurers to enable proactive interventions. By ensuring compliance with data privacy regulations, the invention maintains individual confidentiality while delivering actionable insights that improve personal and public health outcomes.

### BRIEF DESCRIPTION OF THE DRAWINGS

No drawings are submitted with the present provisional application. The invention is fully described and enabled through the written specification, which defines the structural and functional relationships of the modular sampling insert architecture independent of any particular geometric depiction. Any prior references to figures in earlier drafts were illustrative only and are not necessary for understanding or practicing the disclosed invention.

### DETAILED DESCRIPTION OF THE INVENTION

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus of the present invention.

The present invention provides a system and method for collecting, analyzing, and utilizing real-time walking and balance data from users via smartphone operating systems (iOS and Android) to deliver both individualized health insights and population-level health trend analysis. This system leverages opt-in mechanisms, advanced data analysis, and secure anonymization to bridge the gap between personal health monitoring and proactive public health management.

The system begins with opt-in data collection, where users voluntarily share their mobility data through native smartphone operating system settings. Using built-in smartphone sensors, such as accelerometers and gyroscopes, the system gathers metrics including gait speed, stride length, balance stability, and fall risk. All data is anonymized and securely stored to comply with privacy regulations, such as GDPR and HIPAA, ensuring user confidentiality.

Once the data is collected, individualized analysis and warnings are provided. The system uses machine learning algorithms to track a user's walking and balance metrics over time. If deviations from normal patterns indicate increased risks, such as a likelihood of falling, the system generates real-time warnings. These warnings are delivered directly to the user, accompanied by actionable recommendations, such as consulting a healthcare provider, adjusting medications, or engaging in physical therapy.

In addition to individual analysis, the system performs population-level data aggregation and trend analysis. Anonymized data from multiple users is processed to identify trends across various demographics, geographic regions, and time periods. For instance, the system may detect an increase in fall risks among older adults in specific locations or seasonal trends in mobility issues. These insights are shared with public health organizations, healthcare providers, and insurers, enabling proactive interventions. Trends such as correlations between medication use and balance instability can also be identified, providing further opportunities for targeted healthcare strategies.

The system also facilitates health alerts to public health entities. For example, an observed increase in fall risk within a particular city might prompt public health campaigns or localized outreach programs for at-risk populations. By delivering these insights in real-time, the invention allows healthcare systems and policymakers to implement preventive measures quickly, reducing long-term healthcare costs and improving population health outcomes.

Through the integration of real-time data collection, advanced analytics, and secure anonymization, this invention not only provides users with personalized health warnings but also supports broader public health efforts. Its dual focus on individual and population-level insights makes it a transformative tool for health monitoring, offering immediate and long-term benefits to users, healthcare providers, and public health systems.

The system is set to run on a computing device or mobile electronic device. A computing device or mobile electronic device on which the present invention can run would be comprised of a CPU, Hard Disk Drive, Keyboard, Monitor, CPU Main Memory and a portion of main memory where the system resides and executes. Any general-purpose computer, smartphone, or other mobile electronic device with an appropriate amount of storage space is suitable for this purpose. Computer and mobile electronic devices like these are well known in the art and are not pertinent to the invention. The system can also be written in a number of different languages and run on a number of different operating systems and platforms.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

Furthermore, other areas of art may benefit from this method and adjustments to the design are anticipated. Thus, the scope of the invention should be determined by the appended claims and their legal equivalents, rather than by the examples given.

The embodiments of the invention in which an exclusive property or privilege is claimed are defined as follows:

1. A non-transitory computer-readable medium storing instructions that, when executed by a processor, enable a system for collecting and analyzing walking and balance data for individualized health monitoring and population-level health analysis, comprising:
a smartphone operating system configured with an opt-in mechanism to collect walking and balance data from users;
sensors within a smartphone, including at least an accelerometer and a gyroscope, configured to measure gait speed, stride length, balance stability, and fall risk;
a communication module configured to transmit the collected data to a secure storage system;
a data processing unit configured to analyze the walking and balance data using machine learning algorithms to identify individual risks and population-level health trends;
a warning system configured to deliver real-time alerts to users regarding individualized health risks based on the analyzed data; and
an anonymization module configured to securely anonymize the data for compliance with privacy regulations before aggregating it for population-level analysis.

2. The system of claim 1, wherein the opt-in mechanism is integrated into a mobile application that allows users to customize data-sharing preferences.

3. The system of claim 1, wherein the warning system provides recommendations to users, including consulting healthcare professionals, adjusting medications, or increasing physical activity.

4. The system of claim 1, wherein the data processing unit uses seasonal trends to identify increased health risks during specific time periods.

5. The system of claim 1, wherein the anonymized data is aggregated to produce region-specific reports for public health organizations.

6. The system of claim 1, further comprising a data visualization interface for healthcare providers to access population-level health trends.

7. The system of claim 1, wherein the data processing unit correlates fall risks with specific demographic factors, including age and geographic location.

8. The system of claim 1, further comprising a feedback loop to update individual risk thresholds based on new data collected from the user.

9. The system of claim 1, wherein the warning system includes a mobile push notification system to deliver real-time health alerts.

10. The system of claim 1, further comprising a secure API for sharing aggregated data with insurers and healthcare providers.

11. A method for collecting and analyzing walking and balance data to provide individualized health warnings and population-level health insights, comprising:
enabling users to opt in to data collection through a smartphone operating system;
collecting walking and balance data using smartphone sensors, including an accelerometer and a gyroscope, to measure gait speed, stride length, balance stability, and fall risk;
transmitting the collected data to a secure storage system;
analyzing the data with a machine learning algorithm to identify individual health risks and population-level trends;
generating real-time health warnings for users when individual metrics exceed predefined thresholds;
anonymizing the collected data for privacy compliance; and
aggregating the anonymized data to produce health trend reports for public health organizations and healthcare providers.

12. The method of claim 11, further comprising providing users with recommendations based on identified risks, including physical activity adjustments or medical consultations.

13. The method of claim 11, wherein the data collection includes periodic sampling to track changes in balance stability over time.

14. The method of claim 11, further comprising identifying seasonal health trends and notifying users and public health entities about increased risks.

15. The method of claim 11, wherein the machine learning algorithm customizes risk thresholds for each user based on historical data.

16. The method of claim 11, further comprising sharing aggregated health trend data with insurers to identify high-risk populations.

17. The method of claim 11, wherein real-time health warnings are delivered to users through push notifications on their smartphones.

18. The method of claim 11, further comprising generating visual analytics for healthcare providers to understand population health trends.

19. The method of claim 11, wherein the anonymization process involves removing personally identifiable information before storing the data.

20. The method of claim 11, further comprising alerting public health organizations when aggregated data indicates emerging health risks in specific regions.
